(19) United States
(12) Patent Application Publication
(10) Pub. No.: US 2007/0010844 A1
(43) Pub. Date: Jan. 11, 2007

(54) Radiopaque expandable body and methods

(76) Inventors: Gorman Gong, Santa Clara, CA (US); Reynaldo A. Osorio, Daly City, CA (US)

Correspondence Address: J. Michael Boggs;Kilpatrick Stockton LLP, 1001 West Fourth Street, Winston-Salem, NC, 27101, US

(21) Appl. No.: 11/177,042

(22) Filed: Jul. 8, 2005

(51) Int. Cl. A61M 29/00

(52) U.S. Cl. 606/192

(57) ABSTRACT

Systems and methods for radioscopic visualization of the positioning of an expandable body in an interior body region are described. One such system includes a radiopaque marking pattern in communication with the expandable body. The radiopaque marking pattern can be configured to allow for visualizing radioscopically the orientation and movement of the expandable body in unexpanded and expanded states in an interior body region. Such a device is useful for diagnostic or therapeutic purposes, including, for example, providing cavities in interior body regions.

### FIELD OF THE INVENTION

The present invention relates to systems and methods for radioscopic visualization of the positioning of an expandable body in an interior body region. Such systems and methods may be useful for diagnostic or therapeutic purposes, for example, creating a cavity in a vertebral body.

### BACKGROUND OF THE INVENTION

Certain diagnostic or therapeutic procedures require provision of a cavity in an interior body region. For example, as disclosed in U.S. Pat. Nos. 4,969,888 and 5,108,404, a balloon may be deployed to form a cavity in cancellous bone tissue as part of a therapeutic procedure that fixes fractures or other abnormal bone conditions. The balloon or other expandable body may compress the cancellous bone to form an interior cavity. A filling material, such as a bone cement, may be inserted into the cavity in order to provide interior structural support for cortical bone.

This procedure can be used to treat cortical bone, which—due to osteoporosis, avascular necrosis, cancer, trauma, or other disease—is fractured or is prone to compression fracture or collapse. These conditions, if not successfully treated, can result in deformities, chronic complications, and an overall adverse impact upon the quality of life.

Once deployed in a target interior body region of a patient, a balloon, which may be attached to the distal end of a catheter, can be manipulated to an expanded geometry. Positioning and orientation of the balloon in the internal body region can be monitored indirectly by use of markings or other externally viewable indicia on the catheter and/or attached fittings. It is desirable to more precisely direct balloon expansion and to monitor balloon integrity during use by monitoring balloon positioning in a more direct manner.

One approach to more directly monitoring balloon movement in an interior body region is by radioscopic visualization. Conventional medical balloons are constructed of materials that are radio-lucent, or translucent to radiation, and thus would not show up under x-ray or fluoroscopy. Instead, a fluid injected through the catheter to expand the balloon can be radiopaque, or opaque to radiation, to facilitate visualization of the balloon under x-ray fluoroscopy.

A disadvantage of using radiopaque contrast media is that accidental exposure to such radiopaque media can cause hypersensitivity reactions in some patients. Other disadvantages include difficulty in handling radiopaque media that are more viscous than, for example, a saline solution, and that radiopaque media are generally more expensive than non-radiopaque fluids.

Alternatively, the balloon may be visualized in an interior body region when filled with a non-radiopaque fluid, such as sterile water or a saline solution, by using magnetic resonance imaging (MRI). However, the availability of MRI monitoring may be limited, and the use of MRI can be very expensive.

### SUMMARY OF THE INVENTION

Embodiments of the present invention provide systems and methods for radioscopic visualization of the positioning of an expandable body in an interior body region. One illustrative embodiment comprises an expandable body and a radiopaque marking pattern in communication with the expandable body. The radiopaque marking pattern can be configured to allow for visualizing radioscopically the positioning of the expandable body in an interior body region.

In an illustrative embodiment, the radiopaque marking pattern can include a plurality of radiopaque markers. Each of the radiopaque markers can be in communication with a predetermined location on the expandable body. As a result, the positioning—for example, the orientation and movement—of the expandable body in various directions can be visualized radioscopically. Such a device is useful for diagnostic or therapeutic purposes, including, for example, providing cavities in interior body regions.

Features of a radiopaque expandable body and method of the present invention may be accomplished singularly, or in combination, in one or more of the embodiments of the present invention. As will be realized by those of skill in the art, many different embodiments of a radiopaque expandable body and method according to the present invention are possible. Additional uses, advantages, and features of the invention are set forth in the detailed description herein and will become more apparent to those skilled in the art upon examination of the following.

### BRIEF DESCRIPTION OF THE DRAWINGS

FIG. 1 is a view of a cannula having an expandable body coupled to one end of an elongate member inserted through the cannula in an embodiment of the present invention.

FIG. 2 is an enlarged side view of the expandable body shown in the embodiment in FIG. 1.

FIG. 3 is an elevation (lateral) view of several human vertebrae, with a cannula establishing a path to a vertebral body of one of the vertebrae.

FIG. 4 is a plan (coronal) view of a human vertebra being accessed by a cannula, with portions of the vertebra removed to reveal cancellous bone within a vertebral body.

FIG. 5 is a plan (coronal) view of the human vertebra shown in FIG. 4, showing an expandable body expanding in a directionally controlled manner in an embodiment of the present invention.

FIG. 6 is a side view of an expandable body having a radiopaque marking pattern laminated between two tubing layers in an embodiment of the present invention.

FIG. 7 is a side view of an expandable body surrounded by an expandable radiopaque cage in unexpanded condition in an embodiment of the present invention.

FIG. 8 a side view of the expandable body surrounded by an expandable radiopaque cage shown in FIG. 7, in expanded condition in an embodiment of the present invention.

FIG. 9 is a flow chart of a method according to an embodiment of the present invention.

FIG. 10 is a flow chart of a method according to another embodiment of the present invention.

### DETAILED DESCRIPTION

Embodiments of the present invention provide systems and methods for radioscopic visualization of the positioning of an expandable body in an interior body region. The systems and methods embodying the invention can be adapted for use in many suitable interior body regions, wherever the formation of a cavity within or adjacent one or more layers of tissue may be required for a therapeutic or diagnostic purpose. The illustrative embodiments show the invention in association with systems and methods used to treat bones. In other embodiments, the present invention may be used in other interior body regions or types of tissues.

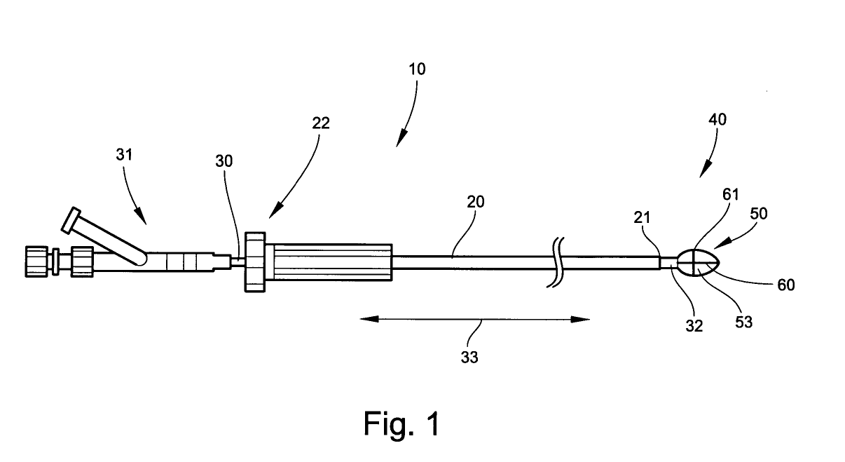

Referring now to the figures, FIG. 1 is a view of a system 10 according to an embodiment of the present invention configured to allow an user to provide a cavity in a targeted treatment area in an interior body region. The system 10 includes an expandable body 50 configured to be used in a kyphoplasty procedure. Kyphoplasty is a minimally invasive surgical procedure for restoring height to, for example, an injured or diseased vertebra. In a kyphoplasty procedure, after a cavity is formed in a vertebral body, a filler material is introduced into the resulting cavity to provide increased height and stability to, for example, the vertebra.

The system 10 comprises a cannula 20 comprising two ends, referred to herein as a proximal end 22 and a distal end 21. The cannula 20 may be fabricated from a material selected to facilitate advancement and rotation of an elongate member 30 movably disposed within the cannula 20. The cannula 20 can be constructed, for example, using standard flexible, medical grade plastic materials, such as vinyl, nylon, polyethylenes, ionomers, polyurethane, and polyethylene tetraphthalate (PET). The cannula 20 can also comprise more rigid materials to impart greater stiffness and thereby aid in its manipulation and torque transmission capabilities. More rigid materials useful for this purpose include stainless steel, nickel-titanium alloys (such as Nitinol), and other metal alloys.

The system 10 shown in FIG. 1 comprises the elongate member 30 movably disposed within the cannula 20. The elongate member 30 may be made from a resilient inert material providing torsion transmission capabilities, for example, stainless steel, a nickel-titanium alloy such as Nitinol, and other suitable metal alloys. In other embodiments, the elongate member 30 may be fashioned from a variety of suitable materials, such as a carbon fiber, a glass, or a flexible material, for example, as a plastic or rubber. In an embodiment comprising a flexible elongate member 30, the elongate member 30 may be formed, for example, from twisted wire filaments, such stainless steel, nickel-titanium alloys (such as Nitinol), and other suitable metal alloys.

The elongate member 30 shown is hollow, allowing for movement of a flowable material, for example, a liquid or a gas, through the elongate member 30. The elongate member 30 may comprise a handle (not shown) at its proximal end 31 to aid in gripping and maneuvering the elongate member 30. For example, in an embodiment, such a handle can be formed from a foam material and secured about the proximal end 31 of the elongate member 30.

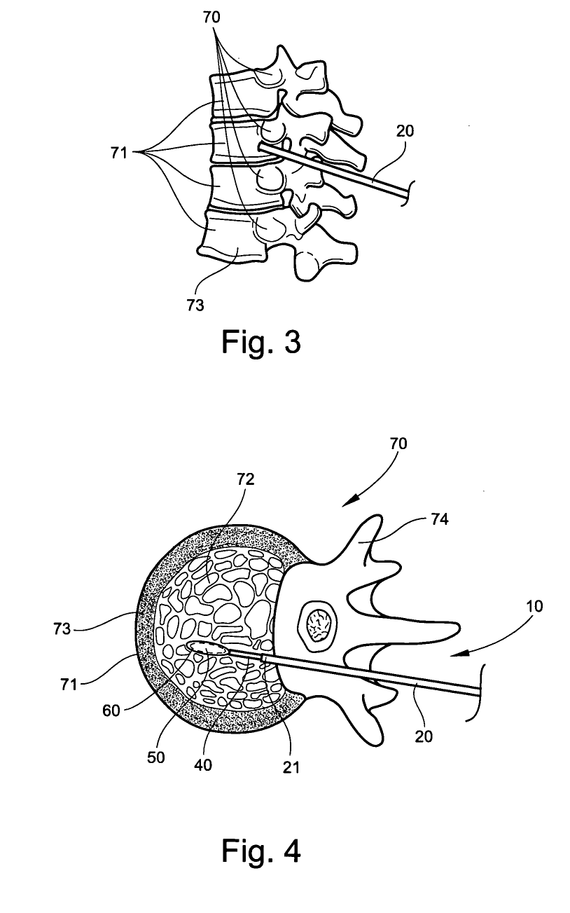

The system 10 shown in FIG. 1 comprises an expandable device 40 that includes an expandable body 50, such as a balloon, configured to be deployed adjacent a tissue in the targeted treatment area via the cannula 20. The expandable body 50 is disposed at the distal end 32 of the elongate member 30, and is thus configured to slide and rotate within the cannula 20. In an embodiment, the expandable body 50 may be configured to be deployed within a treatment area through a percutaneous path established by the cannula 20. For example, the expandable body 50 may be deployed within cancellous bone tissue 72 in a vertebral body 71, as shown in FIGS. 3-5.

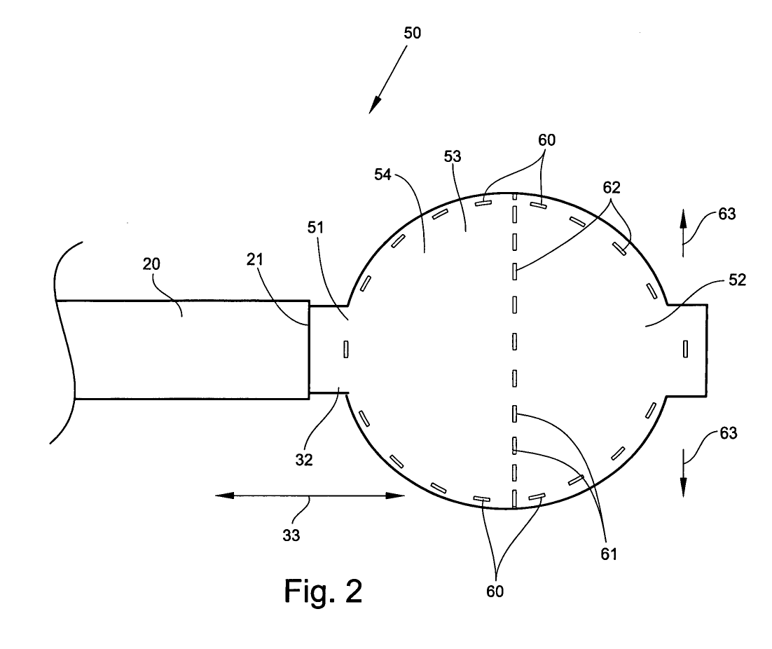

The expandable body 50 may be expanded by movement of a flowable material through the hollow elongate member 30 and into the interior of the expandable body 50. In the embodiment shown in FIGS. 1-2, once the expandable body 50 has been inserted through the cannula 20 to a point beyond the distal end 32 of the cannula 30, a flowable material is introduced through the elongate member 30 to expand the expandable body 50. The expandable body 50 may be contracted by withdrawing the flowable material out of the expandable body 50 through the bore of the elongate member 30. The elongate member 30 and the contracted expandable body 50 may then be withdrawn through the cannula 20.

As shown in FIGS. 1 and 2-8, the expandable body 50 includes a radiopaque marking pattern 60. The radiopaque marking pattern 60 is configured to allow for visualizing radioscopically the positioning, for example, movement and orientation, of the expandable body 50 in the interior body region 70. In this manner, the user can directly monitor positioning of the expandable body 50 in the interior body region 70 while expanding the expandable body 50 by introducing a flowable material that is a non-radiopaque contrast medium. As a result, the risk of exposing a patient to such a radiopaque contrast agent is eliminated.

Radiopaque is defined as being opaque to radiation and especially x-rays. Radioscopy is defined as examination of the inner structure of optically opaque objects by x-rays or other penetrating radiation. Fluoroscopy is defined as examination by means of a fluoroscope. A fluoroscope is a device equipped with a fluorescent screen on which the internal structures of an optically opaque object, such as the human body, may be viewed as shadowy images formed by the differential transmission of x-rays through the object.

In the embodiment shown in FIGS. 1-2, the system 10, and in particular the expandable body 50, may be used to provide a cavity in the interior body region 70. A user of the system 10 causes the expandable body 50 to expand and provide force to surrounding tissues to create a cavity of a desired shape and dimension.

Once a cavity is created in the target treatment area, the expandable body 50 may be contracted and removed from the interior body region 70 through the cannula 20. After the expandable body 50 is removed, a material or filler, such as a bone cement, may then be used to fill the cavity provided by the system 10. Use of a filler material may be beneficial in certain treatment areas, for example, in a vertebra 70 where the system 10 is used to restore height to a vertebral body 71.

Referring now to FIGS. 3-4, an elevation (lateral) view of several human vertebrae 70 is shown, with a cannula 20 establishing a percutaneous path along its longitudinal axis 33 to a vertebral body 71 of one of the several vertebrae 70. The vertebral body 71 extends on the anterior (i.e., front or chest) side of the vertebrae 70. The vertebral body 71 comprises an exterior formed from compact cortical bone 73. Cortical bone (73) is defined as bone consisting of, or relating to, cortex, or outer layer of a bony structure. The cortical bone 73 encloses an interior volume of reticulated cancellous 72, or spongy, bone (also called medullary bone or trabecular bone). Cancellous bone (72) is defined as bone having a porous structure having many small cavities or cells in it.

Due to various traumatic or pathologic conditions, such as osteoporosis, a vertebral body can experience a vertebral compression fracture (VCF). In such conditions, cancellous bone 72 can be compacted, causing a decrease in height of the vertebra 70. In a VCF in particular, vertebral height is lost in the anterior region of the vertebral body 71. The user of the system 10 may utilize it to provide a cavity within the vertebral body 71, and to restore height to the vertebral body 71 lost when a fracture occurred.

Systems and methods according to the present invention are not limited in application to human vertebrae 70, and may be used to provide cavities within other parts of a living or non-living organism. For example, in embodiments, the system 10 can be deployed in other bone types and within or adjacent other tissue types, such as in a vertebral disc, an arm bone, a leg bone, a knee joint, etc.

The vertebral body 71 is generally in the shape of an oval disc. As FIGS. 3-4 show, access to the interior volume of the vertebral body 71 can be achieved, for example, by drilling an access portal through a rear side of the vertebral body 71 (a postero-lateral approach). The portal for the postero-lateral approach enters at a posterior side of the vertebral body 71 and extends anteriorly into the vertebral body 71. Alternatively, access into the interior volume of a vertebral body 71 can be accomplished by drilling an access portal through one or both pedicles 74 of the vertebra 70. This is known as a transpedicular approach.

FIG. 4 shows a vertebra 70 being accessed by the system 10 according to an embodiment of the present invention. The vertebra 70 is shown with portions removed to reveal cancellous bone 72 within the vertebral body 71. The user of the system 10 may slide the elongate member 30 and expandable body 50 axially, or lengthwise along the longitudinal axis 33, within the cannula 30 to deploy the expandable body 50 in the targeted treatment site. When deployed at the site, the user can extend the expandable body 50 outside the distal end 32 of the cannula 20 adjacent cancellous bone tissue 72 within the vertebral body 71. The user may rotate the elongate member 30, and thereby the expandable body 50, to position the expandable body 50 for directed expansion in the targeted treatment area. Once moved beyond the distal end 21 of the cannula 20, the expandable body 50 may be expanded from a contracted state to an expanded state to provide a cavity within the cancellous bone 72.

In embodiments of the present invention, as shown, for example, in FIG. 2, the radiopaque marking pattern 60, 61 comprises a plurality of radiopaque markers 62. Each of the radiopaque markers 62 is in communication with a predetermined location on the expandable body 50. For example, a radiopaque marker 62 can be placed at or near the distal end 52 of the expandable body 50 to indicate the most distal reach of the expandable body 50. A radiopaque marker 62 can be placed at or near the proximal portion 51 of the expandable body 50 (attached to the elongate member 30 or catheter) to indicate the most proximal location of the expandable body 50 when viewed radioscopically. Radiopaque markers 62 can be placed at spaced apart locations on the expandable body 50. In this manner, when the expandable body 50 is expanded, the positioning of the wall 53 of the expandable body 50 can be observed radioscopically.

Positioning of the expandable body 50 in the interior body region 70 can include movement that occurs during and after insertion, anterior-posterior movement along the longitudinal axis 33 of the insertion device (cannula 20), rotating about a radial axis 63, movement of all or a portion of the wall 53 of the expandable body 50 during expansion and/or contraction, and any other orienting or movement of the expandable body 50 capable of being observed by radioscopic visualization of the radiopaque marking pattern 60, 61.

Certain embodiments of the present invention for visualizing the expandable body 50 under fluoroscopy provide the advantage of avoiding the need to inject a radiopaque contrast medium into the expandable body 50. Because an operator can visualize the expandable body 50 without injecting a contrast medium, a non-radiopaque fluid such as a 0.9% sodium chloride solution (normal saline) can be injected into the expandable body 50 to expand the body 50. This avoids any risk to a patient of rupture of the expandable body 50 and leakage of radiopaque contrast media into tissue. This is a particular advantage to persons having a hypersensitivity to contrast media. In addition, saline solution is less viscous and easier to handle than contrast media. Saline solution is also much less costly than contrast media.

Embodiments of the present invention can include the radiopaque marking patterns 60, 61 at various predetermined locations on the expandable body 50. For example, the expandable body 50 can include radiopaque markers 62 in the radiopaque marking pattern 60 (longitudinal pattern) along the longitudinal axis 33 of the expandable body 50, as shown in FIGS. 2, and 4-6. As a result, movement of the expandable body 50 along the longitudinal axis 33 and expansion of the expandable body 50 along the longitudinal axis 33 can be visualized radioscopically.

In an embodiment, the expandable body 50 can include radiopaque markers 62 in the radiopaque marking pattern 61 (radial pattern) about the radial axis 63 of the expandable body 50, as shown in FIG. 2. Accordingly, rotation of the expandable body 50 and radially outward expansion of the expandable body 50 can be visualized radioscopically. This allows the user to use an appropriate amount of rotational torque to properly orient the expandable body 50 in directions desired for optimal effect on targeted tissue when the expandable body 50 is expanded.

In an embodiment, the expandable body 50 can include radiopaque markers 62 located about the periphery 54 of the expandable body 50. Such placement of the radiopaque markers 62 during manufacture of the expandable body 50 provides essentially an outline of the shape of the expandable body 50 when expanded. As such, when the expandable body 50 is expanded, the periphery 54 of the expanded expandable body 50, and thereby the outer contact points of the body 50 onto tissue in the interior body region 70 can be visualized radioscopically.

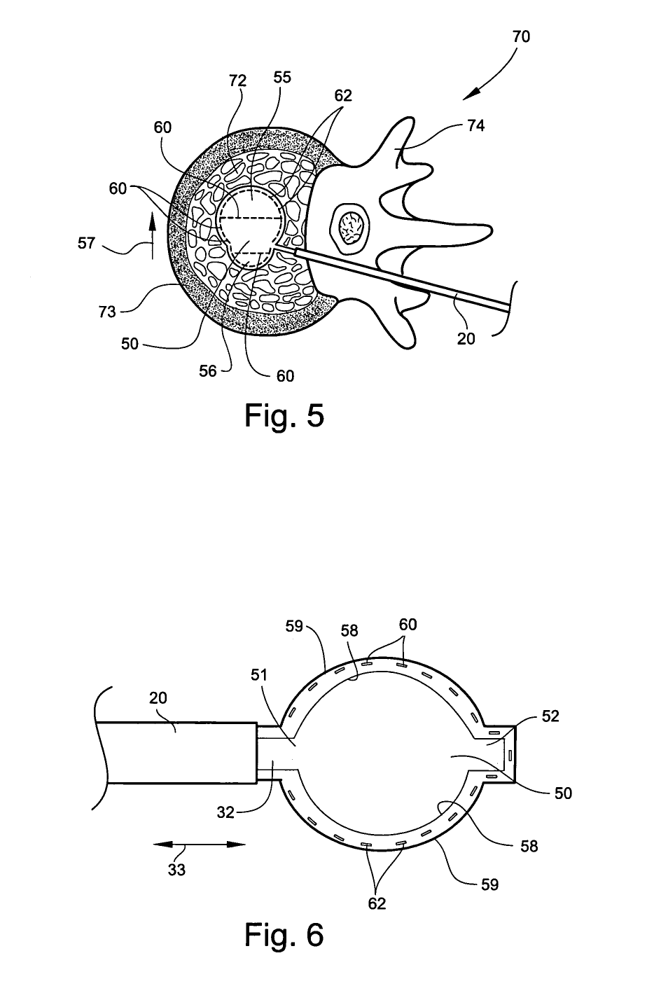

In embodiments of the present invention, the expandable body 50 can be configured to provide directionally-controlled expansion. For example, as shown in the embodiment in FIG. 5, the expandable body 50 can comprise a wall 53 having a higher elasticity, or relatively more compliant, wall portion 55 and a lower elasticity, or relatively less compliant, wall portion 56. Upon expansion of the expandable body wall 53, movement of the lower elasticity wall portion 56 is constrained more than movement of the higher elasticity wall portion 55. As a result, expansion of the expandable body 50 can be directed in the direction 57 of expansion outwardly from the longitudinal axis 33 of the expandable body 50 toward a desired target area. In embodiments, the radiopaque marking pattern 60 can be located in communication with the higher elasticity wall portion 55 so that movement of the higher elasticity wall portion 55 can be visualized radioscopically. In another embodiment, the radiopaque marking pattern 60 can be located in communication with the higher elasticity wall portion 55, and a different radiopaque marking pattern 60 can be located in communication with the lower elasticity wall portion 56. In this configuration, movement of the higher elasticity wall portion 55 relative to the lower elasticity wall portion 56 can be visualized radioscopically.

In such embodiments providing directionally-controlled expansion, movement of the portions of the expandable body wall 53 having different levels of elasticity, or compliance, can be visualized under fluoroscopy as those portions 55, 56 are expanded to directionally-controlled locations in a treatment area, for example, in the vertebral body 71. Radiopaque marker 62 materials can be located on those portions 55, 56 of the expandable body wall 53 such that as the portions 55, 56 are being expanded, the periphery 54 of the expandable body 50 can be observed as it expands. In this manner, an operator can visualize the outer periphery, or border, 54 of expandable body 50 expansion. Accordingly, an operator can be provided with direct, real-time feedback as to both directionality and the degree of expandable body 50 expansion. Visualization of expandable body 50 movement under fluoroscopy allows more accurate observation of the degree of expandable body 50 expansion than merely monitoring the amount of fluid, such as normal saline, injected into the expandable body 50.

In embodiments of the present invention, in addition to an expandable body 50 including a radiopaque marking pattern 60, 61, a deployment device, such as the elongate member 30, can include radiopaque material(s) or markers 62. In this manner, positioning of the deployment device itself can be visualized radioscopically. As a result, the user can monitor positioning of the entire deployment device-expandable body device and any differences in positioning of one component relative to the other component.

Embodiments of the present invention can include methods of making the expandable body 50 having the radiopaque marking pattern 60, 61. For example, as shown in FIG. 6, the expandable body 50 can comprise an inner tubing layer 58 and an outer tubing layer 59. The radiopaque markers 62 can be laminated between the inner and outer tubing layers 58, 59, respectively, to form the radiopaque marking pattern 60. The radiopaque marking pattern 60 allows visualization under fluoroscopy of the positioning of the expandable body 50, progress of expansion of the expandable body 50, and other orientation and/or movement of the expandable body 50. As a result, the need for the fluid to be injected for expanding the expandable body 50 to be a radiopaque contrast medium is eliminated.

In an embodiment of such a laminated expandable body 50, a radiopaque material, or markers, 62, such as stainless steel, can be positioned onto the inner tubing layer 58. The radiopaque markers 62 can be in the form of slivers, shavings, or flakes of the radiopaque material. The outer tubing layer 59 is applied over the inner tubing layer 58 and radiopaque material applied to the inner tubing layer 58. The two tubing layers 58, 59 are then placed into a mold and processed into an embodiment of the expandable body 50 in the form of a balloon. In this manner, the radiopaque markers 62 are encapsulated in position between two layers 58, 59.

The radiopaque markers 62 are preferably adhered to the inner tubing layer 58 so that when the outer tubing layer 59 is applied to the inner tubing layer 58, the markers 62 remain in stable contact with, and do not move relative to, the inner tubing layer 58. Stabilization of the markers 62 on the inner tubing layer 58 prior to applying the outer tubing layer 59 assures that the markers 62 are located in the final two-layered, laminated expandable body 50 in locations desired for monitoring positioning and movement of the expandable body 50 when it is expanded.

The outer tubing layer 59 can be extruded separately from the inner tubing layer 58. The outer tubing layer 59 has a larger inside diameter than the outside diameter of the inner tubing layer 58. The difference in the inside diameter of the outer tubing layer 59 and the outside diameter of the inner tubing layer 58 should be a tolerance that allows the outer tubing layer 59 to be easily applied to the inner tubing layer 58. Such tolerance, or difference in the inside diameter of the outer tubing layer 59 and the outside diameter of the inner tubing layer 58, can be small, for example, approximately 7-8/1000 ths of an inch.

The outer tubing layer 59 can be applied to the inner tubing layer 58 by various methods. One such method includes manually forcing the outer tubing layer 59 over the inner tubing layer 58. The outer tubing layer 59 can be applied to the inner tubing layer 58 by sliding or rolling the outer tubing layer 59 over the inner tubing layer 58. Alternatively, the outer tubing layer 59 can be applied to the inner tubing layer 58 by automated mechanical means.

In an embodiment of such a method of making the laminated expandable body 50 shown in FIG. 6, the two tubing layers 58, 59 having the radiopaque markers 62 enclosed between the layers 58, 59 are placed in a mold. The mold is clamped in position such that the two tubing layers 58, 59 do not move relative to each other. The laminated tubing layers 58, 59 are heated to soften the tubing material. Pressure is then exerted from the inside, or bore, of the inner tubing layer 58 onto the circumference of the two concentric tubing layers 58, 59 toward the walls of the mold. This pressurization process, known as “blowing,” causes the two layers 58, 59 to be permanently bonded together and the radiopaque markers 62 to be sealed in position between the layers 58, 59.

The radiopaque markers 62 can be applied to the expandable body wall 53 in various ways. For example, in a two-layered, laminated expandable body 50 as described, the radiopaque markers 62 can be sprayed onto the inner tubing layer 58. Spraying the radiopaque markers 62 in solution allows the markers 62 to readily adhere to the inner tubing layer 58 in desired locations and prevents uncontrolled disbursement of the markers 62 on the inner tubing layer 58. In an illustrative embodiment, the radiopaque markers 62 can be placed in a solution of a polyurethane, such as TEXIN®, and sprayed onto an inner tubing layer of TEXIN®. This technique allows the radiopaque markers 62 to reliably adhere to the inner tubing layer 58 in controlled locations.

In embodiments, the radiopaque markers 62 can be applied to the expandable body wall 53 by printing, such as with an ink jet printer, or the radiopaque markers 62 can be brushed onto the expandable body wall 53 in desired locations. In another embodiment, the radiopaque markers 62 comprising the radiopaque marking patterns 60, 61 can be adhered to the expandable body wall 53 with an adhesive. In another embodiment, the radiopaque markers 62 can be molded onto the expandable body wall 53. In yet another embodiment, the radiopaque markers 62 can be mixed with an expandable body material, and the expandable body 50 is extruded from that material such that the radiopaque markers 62 form the radiopaque marking patterns 60, 61 as desired in the resulting expandable body 50.

The material(s) used in the expandable body wall 53 can be selected according to the therapeutic objectives surrounding its use. If desired, the material for the expandable body wall 53 can be selected to exhibit generally elastic properties, for example, latex. Alternatively, the material can be selected to exhibit less elastic properties, for example, silicone. During use, the physician monitors expansion of expandable bodies 50 with generally elastic or generally semi-elastic properties to assure that over-expansion and wall 53 failure do not occur. Accordingly, it is important to monitor the movement of expandable bodies 50 during use directly, such as under fluoroscopy.

Expandable bodies 50 of the present invention can be made from, for example, polyurethanes, polyolefins (polyethylenes, polypropylenes, etc.), polyamides, acrylics, polyvinyl compounds, polyesters, polyethers, polycarbonates, polyether terephthalate, polyketones, and any of these materials combined with a filler. Embodiments of expandable bodies 50 according to the present invention can comprise walls 53 made from a single material or from a combination of materials. Such materials can have varying degrees of stiffness or elasticity characteristics. An example of a stiffer, less elastic material useful for making expandable body wall 53 portions is PEBAXT™, a polyether block amide available commercially from Archema. Other engineered plastics may be used. In embodiments, nanocomposites of such materials can be advantageously utilized in the wall 53 of expandable body 50 to decrease elasticity characteristics in the entire wall or in one or more selected portions of the wall. Such materials can also include filler materials and irradiation crosslinked resins.

A less stiff, more elastic material useful for making the wall 53 of expandable body 50 is the polyurethane TEXIN®, commercially available from Bayer MaterialScience in South Deerfield, Mass. Other materials such as silicone, rubber, thermoplastic rubbers, elastomers, and other medical balloon materials can be utilized to make less stiff, more elastic expandable body walls 53.

In one embodiment of the present invention, each of the expandable body 50 and the radiopaque marking patterns 60, 61 comprise a radiopaque material. The radiopaque material of the radiopaque marking patterns 60, 61 can be radioscopically visibly distinct from the radiopaque material of the expandable body 50. Such an embodiment allows radioscopic visualization of the positioning of different radiopaque materials, and thus different portions of the expandable body 50 relative to other portions of the expandable body 50.

In embodiments of the present invention, the radiopaque markers 62 can be made from radiopaque materials including, for example, stainless steel, platinum, gold, calcium, tantalum, and other heavy metals. Radiopaque fillers useful in the radiopaque markers 62 can include, for example, barium sulfate, tantalum, tungsten, and bismuth subcarbonate, among other materials.

The radiopaque material or markers 62 utilized in embodiments of the present invention are sufficiently large to be readily visualized under fluoroscopy. The size of the pieces of radiopaque material used also depends on the manner in which the radiopaque material is applied to the expandable body wall 53. For example, if the radiopaque material or markers 62 are sprayed onto expandable body wall 53, the markers 62 should be small enough to be aerosolized through a spraying device.

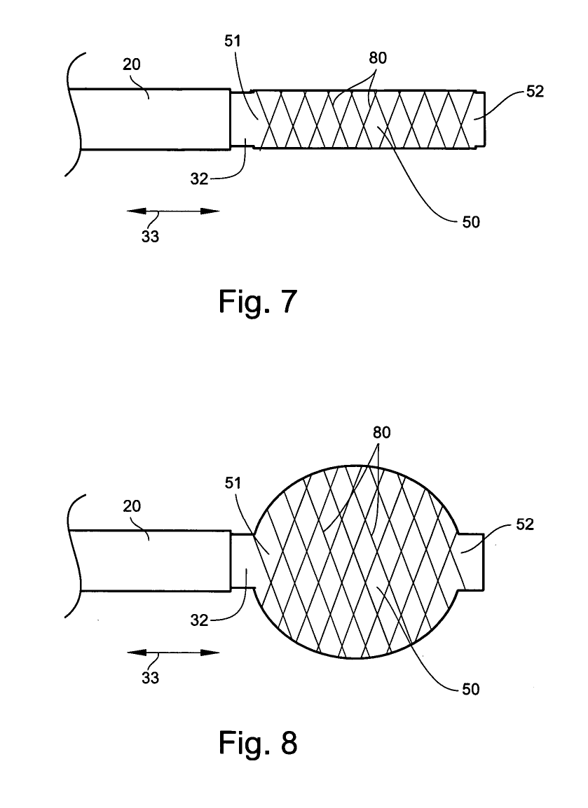

In an embodiment of the present invention, as shown in FIGS. 7-8, the radiopaque marking patterns 60, 61 comprise an expandable radiopaque cage 80 surrounding the expandable body 50. The cage 80 is expandable in response to a pressure exerted by expanding the expandable body 50. When expanded, as shown in FIG. 8, the cage 80 marks an expanded periphery 54 of the expandable body 50. As a result, the expanded periphery 54 of the expandable body 50 can be visualized radioscopically. In operation, as the expandable body 50 is expanded, the cage 80 expands around the periphery 54 of the expandable body 50 to provide essentially an outline of the borders, or periphery, 54 of the expandable body 50. The radiopaque cage 80 allows visualization under fluoroscopy of the positioning of the expandable body 50, progress of expansion expandable body 50, and other movement of the expandable body 50. As a result, the need for the fluid to be injected for expanding the expandable body 50 to be a radiopaque contrast medium is eliminated.

In an embodiment, the radiopaque cage 80 surrounding the expandable body 50 is non-structural, meaning that the cage 80 does not have a primary mechanical function. Because the cage 80 can be made to serve no primary structural purpose, it can be constructed from a less expensive material than if it were structurally functional. Alternatively, the radiopaque cage 80 can provide a structural mechanical function during or after its expansion around the expandable body 50.

In an embodiment, the radiopaque cage 80 is adapted to remain expanded in the interior body region 70 after the expandable body 50 is contracted and removed from the interior body region 70. As such, the cage 80 can be manufactured with biocompatible materials to be an implantable device. The radiopaque cage 80 is designed so that a filler material can be injected about the expanded cage 80 in the interior body region 70. When the radiopaque cage 80 is in position in a bony treatment area, for example, the vertebral body 71, a bone cement, such as polymethyl methacralate (PMMA), can be injected into the area. The bone cement solidifies around the cage 80, and holds the cage 80 permanently in place.

In an alternative embodiment, the expandable radiopaque cage 80 can be removed from the interior body region 70. Removal of the cage 80 can be accomplished, for example, by forming the cage 80 from a material that is collapsible after being expanded. When the expandable body 50 is deflated, the cage 80 structure can collapse back to its original shape such that it can be retracted from the interior body region 70 via the deployment cannula 20. To remove the cage 80, the deployment cannula 20 would accommodate removal of the cage 80 along with the deflated expandable body 50.

Embodiments of the radiopaque cage 80 can be made from the same or similar materials that are used for angioplasty stents and other implantable devices. For example, the radiopaque cage 80 can be made from stainless steel, nickel, titanium, or other radiopaque biocompatible material. The radiopaque cage 80 can also comprise plastics, ceramics, or fibers. Such materials can have the capability of maintaining their expanded shape and thus remain in expanded position in the treatment area of the interior body region 70. In addition, shape-memory alloys, such as the nickel-titanium alloy Nitinol, can be used.

Embodiments of the radiopaque cage 80 can be made in the form of a metal netting, welded metal wires, and/or expandable slit metal tubing. A radiopaque metal netting cage can be in the form of a fine mesh netting formed, for example, from a foamed polymer sheath material. In another embodiment, a radiopaque metal netting cage can be in the form of a fine mesh netting formed from a radiopaque, non-structural, compliant polymer sheath. The cage 80 is made in such a manner as to be expandable in response to the pressure exerted by expanding the expandable body 50.

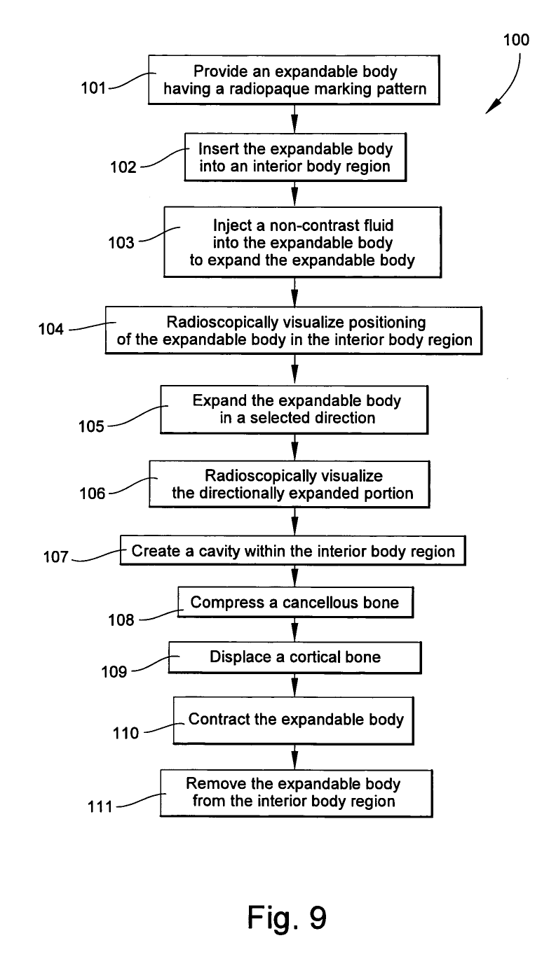

Embodiments of the present invention include methods for radioscopically visualizing positioning of the expandable body 50 in the interior body region 70. As shown in the embodiment of the method 100 in FIG. 9, the expandable body 50 is provided (101) with the radiopaque marking pattern 60, 61. The expandable body 50 is inserted (102) into the interior body region 70. A non-contrast fluid can then be injected (103) into the expandable body 50 to expand it. Positioning of the expandable body 50 in the interior body region 70 can be visualized (104) radioscopically, both before and after expansion with the injected fluid.

In an embodiment, the expandable body 50 can be expanded in a selected direction (105), and the directionally expanded portion of the expandable body 50 can be visualized radioscopically (106). In embodiments, expansion of the expandable body 50 according to the present invention can be utilized to create (107) a cavity within the interior body region 70. In embodiments, expansion of the expandable body 50 can be utilized to compress (108) cancellous bone 72 and/or to displace (109) cortical bone 73. Once the expandable body 50 has been expanded, and utilized for the intended purpose, the expandable body 50 can be contracted (110) and removed (111) from the interior body region 70.

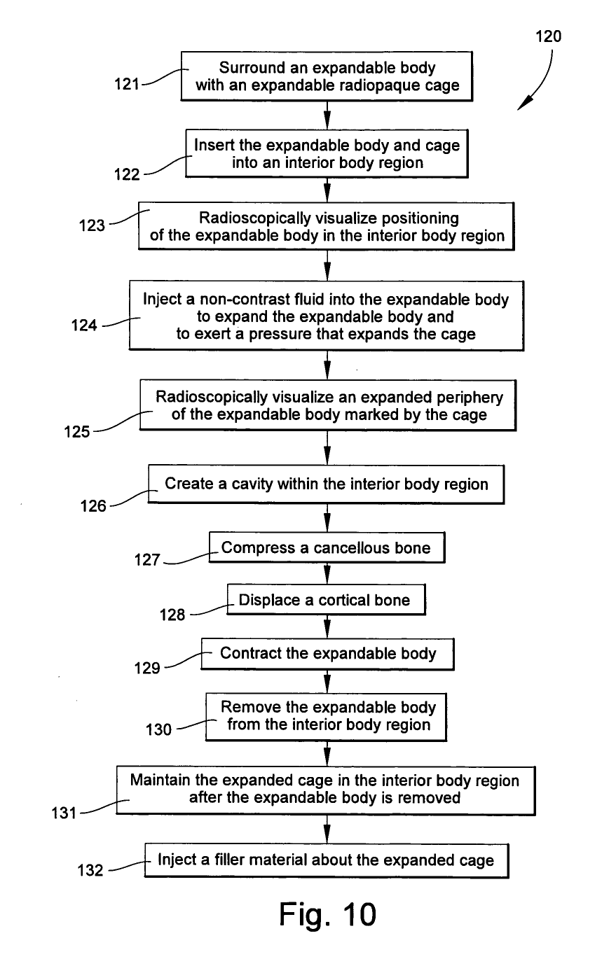

As shown in FIG. 10, the embodiment of the method 120 of the present invention includes providing the expandable body 50 surrounded (121) with the expandable radiopaque cage 80. The expandable body 50 and radiopaque cage 80 are inserted (122) into the interior body region 70, where positioning of the radiopaque cage 80, and thereby the expandable body 50 (both in unexpanded condition), can be visualized radioscopically (123). A non-contrast fluid is then injected (124) into the expandable body 50 to expand the expandable body 50 and to exert a pressure that expands the cage 80. The expanded periphery 54 of the expandable body 50 marked by the cage 80 can then be visualized radioscopically (125).

In embodiments of the present invention, expansion of the expandable body 50 and radiopaque cage 80 can be utilized to create (126) a cavity within the interior body region 70. In embodiments, expansion of the expandable body 50 and radiopaque cage 80 can be utilized to compress (127) cancellous bone 72 and/or to displace (128) cortical bone 73.

Once the expandable body 50 and radiopaque cage 80 have been expanded, and utilized for the intended purpose, the expandable body 50 can be contracted (129) and removed (130) from the interior body region 70. In embodiments, the expanded cage 80 can be maintained (131) in the interior body region 70 after the expandable body 50 is removed (130). A filler material can be injected (132) about the expanded cage 80. In an alternative embodiment, the radiopaque cage 80 can also be contracted and removed from the interior body region 70.

Although the present invention has been described with reference to particular embodiments, it should be recognized that these embodiments are merely illustrative of the principles of the present invention. Those of ordinary skill in the art will appreciate that a radiopaque expandable body and method of the present invention may be constructed and implemented in other ways and embodiments. Accordingly, the description herein should not be read as limiting the present invention, as other embodiments also fall within the scope of the present invention.

What is claimed is:

1. A system comprising:
an expandable body; and
a radiopaque marking pattern in communication with the expandable body and configured to allow for radioscopic visualization of positioning of the expandable body in an interior body region.

2. The system of claim 1, wherein the radiopaque marking pattern comprises a plurality of radiopaque markers, and each of the radiopaque markers is in communication with a predetermined location on the expandable body.

3. The system of claim 1, wherein the radiopaque marking pattern is located radially about the expandable body so that radial movement of the expandable body and radially outward expansion of the expandable body can be visualized radioscopically.

4. The system of claim 1, wherein the radiopaque marking pattern is located along a longitudinal axis of the expandable body so that movement of the expandable body along the longitudinal axis and expansion of the expandable body along the longitudinal axis can be visualized radioscopically.

5. The system of claim 1, wherein the radiopaque marking pattern is located on the expandable body so that when the expandable body is expanded, a periphery of the expanded expandable body can be visualized radioscopically.

6. The system of claim 1, the expandable body comprising a wall having a higher elasticity wall portion and a lower elasticity wall portion, wherein the radiopaque marking pattern is in communication with the higher elasticity wall portion so that movement of the higher elasticity wall portion can be visualized radioscopically.

7. The system of claim 6,
wherein the radiopaque marking pattern in communication with the higher elasticity wall portion comprises a first radiopaque marking pattern, and a second radiopaque marking pattern different from the first radiopaque marking pattern is in communication with the lower elasticity wall portion, and
wherein movement of the higher elasticity wall portion relative to the lower elasticity wall portion can be visualized radioscopically.

8. The system of claim 1, wherein the expandable body comprises a first radiopaque material and the radiopaque marking pattern comprises a second radiopaque material, and wherein the first and second radiopaque materials are radioscopically visibly distinct.

9. The system of claim 1, wherein the expandable body comprises an expandable balloon device.

10. The system of claim 1, wherein the expandable body comprises an inner tubing layer and an outer tubing layer, and wherein the radiopaque marking pattern is laminated between the inner and outer tubing layers.

11. The system of claim 1,
wherein the radiopaque marking pattern comprises an expandable radiopaque cage surrounding the expandable body,
wherein the cage is expandable in response to a pressure exerted by expanding the expandable body, and
wherein, when expanded, the cage marks an expanded periphery of the expandable body.

12. The system of claim 11, wherein the radiopaque cage is adapted to remain expanded in the interior body region after the expandable body is contracted.

13. The system of claim 12, further comprising a filler material injected about the expanded cage.

14. A method, comprising:
applying a radiopaque marking pattern to an expandable body; and
configuring the radiopaque marking pattern to allow for radioscopic visualization of positioning of the expandable body in an interior body region.

15. The method of claim 14, the radiopaque marking pattern comprising a plurality of radiopaque markers, wherein configuring the radiopaque marking pattern comprises placing each of the radiopaque markers in a predetermined location on the expandable body.

16. The method of claim 14, further comprising configuring the radiopaque marking pattern so that when the expandable body is expanded, a periphery of the expanded expandable body can be visualized radioscopically.

17. The method of claim 14, wherein the expandable body is expandable in a selected direction, the method further comprising configuring the radiopaque marking pattern so that a directionally expanded portion of the expandable body can be visualized radioscopically.

18. The method of claim 14, the expandable body comprising an inner layer and an outer layer, the method further comprising
applying the radiopaque marking pattern to the inner layer;
applying the outer layer over the inner layer and the radiopaque marking pattern; and
molding the outer layer, the radiopaque marking pattern, and the inner layer into a laminated body.

19. The method of claim 14, wherein applying the radiopaque marking pattern to the expandable body comprises spraying the radiopaque marking pattern onto the expandable body.

20. The method of claim 14, wherein applying the radiopaque marking pattern to the expandable body comprises applying the radiopaque marking pattern to a material, the method further comprising extruding the expandable body from the material.

21. The method of claim 14,
wherein applying the radiopaque marking pattern to the expandable body comprises surrounding the expandable body with an expandable radiopaque cage,
wherein the cage is expandable in response to a pressure exerted by expanding the expandable body, and
wherein, when expanded, the cage marks an expanded periphery of the expandable body.

22. A method comprising:
inserting into an interior body region an expandable body having a radiopaque marking pattern in communication with the expandable body;
expanding the expandable body; and
radioscopically visualizing positioning of the expandable body in the interior body region.

23. The method of claim 22, wherein expanding the expandable body comprises injecting a non-contrast fluid into the expandable body.

24. The method of claim 22, wherein expanding the expandable body further comprises expanding the expandable body in a selected direction, and wherein the radiopaque marking pattern is configured so that a directionally expanded portion of the expandable body can be visualized radioscopically.

25. The method of claim 22, wherein expanding the expandable body further comprises creating a cavity within the interior body region.

26. The method of claim 25, wherein expanding the expandable body further comprises compressing a cancellous bone.

27. The method of claim 25, wherein expanding the expandable body further comprises displacing a cortical bone.

28. The method of claim 22, further comprising contracting the expandable body and removing the expandable body from the interior body region.

29. The method of claim 22,
wherein the radiopaque marking pattern comprises an expandable radiopaque cage surrounding the expandable body,
wherein expanding the expandable body exerts a pressure that expands the cage, and
wherein an expanded periphery of the expandable body marked by the cage can be visualized radioscopically.

30. The method of claim 29, further comprising removing the expandable body from the interior body region and maintaining the expanded cage in the interior body region after the expandable body is removed.

31. The method of claim 30, further comprising injecting a filler material about the expanded cage.

32. The method of claim 30, further comprising removing the cage from the interior body region.
